(19) United States
(12) Patent Application Publication
(10) Pub. No.: US 2008/0279951 A1
(43) Pub. Date: Nov. 13, 2008

(54) Stable Oral Benzimidazole Compositions Prepared by Non-Aqueous Layering Process

(76) Inventors: Rajesh Gandhi, Haryana (IN); Ramesh Muttavarpu, Andhra Pradesh (IN); Venkata Ram Mohan Rao Visinigiri, Andhra Pradesh (IN); Vishnubhotla Nagaprasad, Andhra Pradesh (IN)

Correspondence Address: RANBAXY INC., 600 COLLEGE ROAD EAST, SUITE 2100, PRINCETON, NJ, 08540, US

(21) Appl. No.: 11/815,160

(22) PCT Filed: Feb. 2, 2006
(86) PCT No.: PCT/IB2006/000194

§ 371 Date: May 8, 2008

(30) Foreign Application Priority Data
Feb. 2, 2005 (IN) 209/DEL/2005

(51) Int. Cl. A61K 9/14; A61K 31/4184; A61K 31/4439

(52) U.S. Cl. 424/490; 514/394; 514/338

(57) ABSTRACT

The present invention relates to processes for the preparation of stable oral compositions of benzimidazole compounds prepared using a non-aqueous layering process.

### FIELD OF THE INVENTION

The present invention relates to processes for the preparation of stable oral compositions of benzimidazole compounds prepared using a non-aqueous layering process.

### BACKGROUND OF THE INVENTION

Benzimidazole compounds, such as omeprazole, lansoprazole, pantoprazole, rabeprazole or single enantiomers thereof are strong inhibitors of proton pump and are widely used as therapeutic agents for stomach ulcers, duodenal ulcers, and gastro esophageal reflux disorders by inhibiting gastric acid secretion.

U.S. Pat. No. 4,255,431 discloses omeprazole and therapeutically acceptable salts thereof. The advantages of providing the salts of omeprazole and particularly the magnesium salt are disclosed in U.S. Pat. No. 4,738,974.

The single isomers of omeprazole are reported to be more useful in therapy when compared to the racemic omeprazole. U.S. Pat. No. 5,877,192 discloses the use of the (−)-enantiomer of omeprazole (esomeprazole), or a pharmaceutically acceptable salt thereof, in the treatment of gastric acid related diseases as a means to decrease interindividual variation in plasma levels when compared to omeprazole. The use of the (−)-enantiomer of omeprazole to receive increased average plasma levels (AUC) of the substance compared to those of racemic omeprazole and thereby a higher dose efficiency is also disclosed in the patent.

U.S. Pat. No. 5,900,424 discloses omeprazole magnesium salt having a degree of crystallinity, which is higher than 70% as determined by X-ray powder diffraction. The patent discloses that the isolation and purification in full manufacturing scale of the magnesium omeprazole salt as per U.S. Pat. No. 4,738,974 presents a major problem. The magnesium omeprazole salt crystals obtained are very fragile. The patent further teaches that in order to use the magnesium salt of omeprazole in a full manufacturing scale in preparing pharmaceutical formulations primarily for oral administration, such as tablets, it is necessary that magnesium omeprazole possesses a combination of properties. These physical properties are degree of crystallinity, particle diameter, density, hygroscopicity, water content and content of other solvents. U.S. Pat. No. 5,690,960 teaches stable oral formulation comprising: a core containing a magnesium salt of omeprazole said salt having more than 70% crystallinity as determined by x-ray powder diffraction; a subcoating layer; and an enteric coating layer.

The efforts to stabilize benzimidazole compositions using amorphous form of benzimidazole compounds are reported in prior art.

WO04/037253 and WO04/002982 teach processes of preparing the amorphous form of a salt of esomeprazole. U.S. Patent Application No. 20030212274 teaches processes of preparing amorphous form of omeprazole salts.

U.S. Pat. No. 6,713,495 discloses magnesium omeprazole having a degree of crystallinity of under 67% by weight and having a residual organic solvent content of less than 7% by weight. U.S. Patent Application No. 20030232861 discloses magnesium S-omeprazole having a degree of crystallinity of under 67%. Example 3 of U.S. Pat. No. 6,713,495 and U.S. Patent Application No. 20030232861 disclose magnesium omeprazole and magnesium esomeprazole respectively, having a degree of crystallinity of under 25%.

Indian Patent Application No. 1494/DEL/2003 teaches stable oral benzimidazole compositions comprising a core comprising an amorphous or crystalline benzimidazole compound, a substantially water-insoluble and substantially non-disintegrating separating layer and an enteric coating.

U.S. Patent Application No. 20020128293 teaches stable oral pharmaceutical composition comprising omeprazole and a stabilizing excipient, wherein the composition is free of alkaline compounds. Example 7 of the patent application discloses a process of wet drug layering of an inert carrier using a wurster fluid bed apparatus.

Because of a strong tendency of benzimidazole compounds to decompose in a neutral and in particular, acidic environment, numerous approaches have been tried to form a stable pharmaceutical formulation. The acid labile benzimidazole compound reacts with both the gastric acid in the stomach and the enteric coatings used for preventing the benzimidazole from coming into contact with the gastric acid.

The prior art teaches various approaches to prepare the stable formulations containing benzimidazole compounds. Amongst the most common approaches was the use of an alkaline core, a separating layer and an enteric coating. It is well-recognized fact that using alkaline medium in the core stabilizes benzimidazole from acid degradation. Prior art efforts to stabilize benzimidazole with an alkaline core without a separating layer between the core and the enteric coating, could not effectively stabilize benzimidazole. Thus, the recognition of use of separating layer to separate the alkaline core with the acidic enteric coating was the subject matter of U.S. Pat. No. 4,786,505 and U.S. Pat. No. 4,853,230. The separating coating disclosed in said patents was essentially made up of water-soluble polymeric substances.

U.S. Pat. No. 5,385,739 discloses a stable formulation of omeprazole microgranules containing a neutral core of sugar and starch and an active layer consisting of a dilution of omeprazole in mannitol in substantially equal amounts. The active omeprazole layer contains about 10% by weight of carboxymethylstarch, about 5% by weight of a sodium lauryl sulfate surface-active compound, and wherein the dilution of omeprazole in mannitol is applied to the neutral core by means of hydroxypropyl methylcellulose as a high viscosity binder.

U.S. Pat. No. 6,159,499 teaches a composition substantially free of alkaline-reacting compounds comprising: (a) a core containing an acid-labile benzimidazole active principle, wherein said core comprises a plurality of nuclei and said active principle mixed together and then compressed together, and wherein said active principle is not in the form of an alkaline salt; (b) an intermediate layer surrounding the core; and (c) an enteric layer surrounding the intermediate layer. The patent uses a process for preparing stable oral dosage form of benzimidazole compounds which are free from alkaline reacting compounds comprising the steps of: (i) mixing a plurality of nuclei with an active principle; (ii) compressing the product of step (i) to form a core comprising the active principle; (iii) coating said core with an intermediate layer; and (iv) coating the product from step (iii) with an enteric layer.

WO99/61022 discloses a stable oral pharmaceutical composition in the form of a mixture containing a substituted pyridyl sulfinyl benzimidazole having gastric acid secretion inhibitory activity and a carrier comprising at least one polymer that is at least partially comprised of vinylpyrrolidone monomeric units. The patent teaches use of mixture of benzimidazole compounds with at least one polymer containing vinylpyrrolidone monomeric units said mixture being free of any alkaline reacting compounds to stabilize benzimidazole compounds. The formulation is in the form of premix that is not compressed to facilitate the manufacturing operations.

U.S. Pat. Nos. 6,274,173; 6,602,522; 5,626,875 and 6,207,198 disclose various other formulation approaches to stabilize benzimidazole compositions.

However, there is still a need for the development of new pharmaceutical compositions of benzimidazole compounds with enhanced stability. Further, it was observed that there are few prior art references with an attempt to prepare stable oral compositions containing amorphous benzimidazole since the decision to use an amorphous drug substance must be made with the knowledge that the amorphous form is metastable relative to crystalline forms. Materials that are visually, or even microscopically, amorphous can be sufficiently ordered at the sub-micron scale to have a diffraction pattern. This suggests that, during the various processing steps of the composition, even an amorphous form may transform to a crystalline phase.

It was surprisingly observed that the preparation of benzimidazole core using a non-aqueous layering process led to a stable amorphous benzimidazole composition. The process substantially prevented the conversion of the amorphous form of benzimidazole compound to the crystalline form.

### SUMMARY OF THE INVENTION

In one general aspect there is provided a process for the preparation of a stable oral benzimidazole composition. The process includes the steps of: a) preparing a benzimidazole core formed by dispersing amorphous benzimidazole compound and one or more pharmaceutically acceptable additives in one or more of non-aqueous solvents to obtain a dispersion and spraying the dispersion on a pharmaceutically acceptable inert carrier; b) coating the core with a separating layer; and c) coating the product of step (b) with an enteric coating, wherein the process of preparing the benzimidazole core substantially prevents the conversion of benzimidazole compound to its crystalline form.

Embodiments of the present invention may include one or more of the following features. For example, the benzimidazole compound comprises one or more of omeprazole, lansoprazole, rabeprazole, pantoprazole, leminoprazole, pariprazole, single enantiomers, pharmaceutically accepted salts, solvates or mixtures thereof. The benzimidazole compound may be omeprazole magnesium. In addition, the stable oral benzimidazole composition may include no more than about 30% by weight of the crystalline benzimidazole compound.

The non-aqueous solvent comprises one or more of methanol, ethanol, isopropanol, methylene chloride, acetone and mixtures thereof.

The pharmaceutically acceptable additive may include one or more of binders, diluents, disintegrants, lubricants and wetting agents. The binder may include one or more of cellulose derivatives, gums, water-soluble vinylpyrrolidone polymers and sugars. The diluent may include one or more of sugars, sugar alcohols, cellulose derivatives, and starches. The disintegrant may include one or more of sodium starch glycolate, croscarmellose sodium, crospovidone, corn starch and mixtures thereof. The lubricant may include one or more of magnesium stearate, talc, sodium stearyl fumarate, colloidal silicon dioxide and mixtures thereof. The wetting agent may include one or more of sodium lauryl sulphate, polysorbate 80 and mixtures thereof.

The separating layer may include one or more substantially water soluble materials. The substantially water-soluble material may include one or both of a substantially water-soluble polymer and a substantially water-soluble excipient. The water-soluble polymer may include one or more of hydroxypropyl methylcellulose, hydroxypropyl cellulose, polyvinylpyrrolidone, sodium alginate, sodium carboxymethyl cellulose, and copolymer of vinylpyrrolidone and vinyl acetate. The water-soluble excipient may include one or more of lactose, mannitol, sorbitol, sucrose and glucose.

### DETAILED DESCRIPTION OF THE INVENTION

The present invention is directed to a process for the preparation of a stable oral benzimidazole composition. The process includes the steps of: a) preparing a benzimidazole core formed by dispersing amorphous benzimidazole compound and one or more pharmaceutically acceptable additives in one or more of non-aqueous solvents to obtain a dispersion and spraying the dispersion on a pharmaceutically acceptable inert carrier; b) coating the core with a separating layer; and c) coating the product of step (b) with an enteric coating, wherein the process of preparing the benzimidazole core substantially prevents the conversion of benzimidazole compound to its crystalline form.

The term ‘benzimidazole compound’ used herein refers to any of the compounds belonging to the category of benzimidazole used for gastrointestinal disorders and may include one or more of omeprazole, lansoprazole, rabeprazole, pantoprazole, leminoprazole and pariprazole, single enantiomers, pharmaceutically accepted salts, solvates or mixtures thereof. For example, the benzimidazole compound may be omeprazole in the form of a pharmaceutically acceptable alkaline salt. The omeprazole may be in the form of omeprazole magnesium. The benzimidazole compound may be in amorphous form. Amorphous omeprazole magnesium may be prepared according to U.S. Patent Application 20030212274 herein incorporated by reference. However any other suitable method can be used to prepare amorphous omeprazole magnesium used in the present invention.

The term ‘stable oral composition’ as used herein refers to the oral compositions of amorphous benzimidazole compounds, which are substantially free from crystalline benzimidazole. Preferably, the stable oral composition contains NMT about 30% by weight of crystalline benzimidazole. The suitable method of determining the conversion of the amorphous form to the crystalline form is any method with substantial precision, for e.g. X-ray diffraction spectroscopy.

The term ‘benzimidazole core’ as used herein comprises amorphous benzimidazole compound and one or more pharmaceutically acceptable additives, which are substantially free from crystalline benzimidazole. Preferably, the benzimidazole core contains NMT about 30% by weight of crystalline benzimidazole. The benzimidazole core is prepared using non-aqueous layering process in order to prevent the conversion of amorphous benzimidazole to crystalline benzimidazole. Preferably, the conversion to crystalline benzimidazole is NMT about 30% by weight. The benzimidazole core may be obtained in the form of granules, pellets, beads or minitablets, which may be further processed to obtain benzimidazole composition in suitable dosage form. For example, the benzimidazole core may be coated with a separating layer and an enteric coating to obtain coated core. The coated core may be filled into capsules or compressed into tablets.

The term ‘composition’ refers to any oral dosage form, such as tablets or capsules, comprising the benzimidazole core.

The non-aqueous layering process uses non-aqueous solvents or mixtures thereof for the preparation of the benzimidazole core. The process substantially prevents the conversion of benzimidazole compound to its crystalline form such that the benzimidazole core contains NMT about 30% by weight of crystalline benzimidazole.

The non-aqueous solvents may include one or more of methanol, ethanol, isopropanol, methylene chloride, acetone or mixtures thereof.

The ‘pharmaceutically acceptable’ additives may include one or more of binders, diluents, disintegrants, lubricants/glidants and solubilizers/wetting agents.

Suitable diluents may include sugars, such as dextrose, glucose, lactose; sugar alcohols, such as sorbitol, xylitol, mannitol; cellulose derivatives, such as powdered cellulose, microcrystalline cellulose; starches, such as corn starch, pregelatinized starch, or maize starch. The preferred range of diluents depends on the type of composition to be prepared. The preferred range is disclosed in the corresponding examples.

Suitable binders include cellulose derivatives such as hydroxypropylmethyl cellulose, hydroxypropyl cellulose, methylcellulose; gums such as xanthan gum, gum acacia, tragacanth; water-soluble vinylpyrrolidone polymers such as polyvinylpyrrolidone, copolymer of vinylpyrrolidone and vinyl acetate; sugars such as sorbitol, mannitol and mixtures thereof. The preferred range of binders depends on the type of composition to be prepared. The preferred range is disclosed in the corresponding examples.

Suitable disintegrants include sodium starch glycolate, croscannellose sodium, crospovidone, corn starch and mixtures thereof. The preferred range of disintegrants depends on the type of composition to be prepared. The preferred range is disclosed in the corresponding examples.

Suitable solubilizers/wetting agents may include sodium lauryl sulphate, polysorbate 80 or mixtures thereof. Suitable lubricant/glidants may include magnesium stearate, talc, sodium stearyl fumarate, colloidal silicon dioxide and mixtures thereof.

The benzimidazole core is prepared using a non-aqueous layering process in order to prevent the conversion to crystalline benzimidazole. The amorphous benzimidazole compound and optionally a pharmaceutically acceptable additive may be dispersed in a non-aqueous solvent or mixtures thereof to obtain a dispersion. The resulting dispersion may be sprayed on pharmaceutically acceptable inert carrier in a fluidized bed apparatus e.g. Wurster coater. The various process parameters during the drug loading process include the benzimidazole dispersion media, the total solid content in the dispersion, the total spraying time for the preparation of the benzimidazole core, the number of prepared lots of the dispersion, the inlet temperature and the bed temperature during the preparation of the benzimidazole core, the drying temperature, and the ratio of omeprazole to the additive in dispersion.

The ‘pharmaceutically acceptable inert carrier’ may comprise a starch, microcrystalline cellulose or sugar sphere such as nonpareil sugar seeds.

The separating layer as used herein refers to the layer that separates the core from the enteric coating. The separating layer is made up of substantially water soluble material, which is capable of dissolving or forming a gel in contact with water. Such material may include substantially water-soluble polymer and/or substantially water-soluble excipients. In the case when the capsule shell acts as separating layer, additional application of a separating layer would be optional. The enteric coating can directly be layered on capsule shell in such case. The substantially water-soluble excipients may include glucose, lactose, mannitol, sorbitol, sucrose, dextrose or such like. The substantially water-soluble polymers may include hydroxypropylmethylcellulose, hydroxypropyl cellulose, polyvinylpyrrolidone, sodium alginate, sodium carboxymethyl cellulose, copolymer of vinylpyrrolidone and vinyl acetate. For example, the polymers may be hydroxypropylmethylcellulose, hydroxypropyl cellulose or polyvinylpyrrolidone. The range of such substantially water-soluble polymers depends on the type of compositions to be prepared.

The enteric coating may include polymers, such as cellulose acetate phthalate, hydroxypropylmethyl cellulose phthalate, polyvinyl acetate phthalate, carboxymethylethylcellulose, methacrylic acid copolymers, such as compounds known under the trademarks of Eudragit NE30D, Eudragit L, Eudragit S, Eudragit L 100 55 or mixtures thereof. The enteric coating may also contain plasticizers, such as triacetin, triethyl citrate, tributyl sebecate, diethyl phthalate, polyethylene glycol and inert excipients, such as talc, titanium dioxide, colloidal silicon dioxide, hydroxypropyl methylcellulose, and crospovidone.

The compositions of the present invention show substantial absence of crystalline benzimidazole when stored at 40° C. and 75% humidity conditions for a period of at least 1 month, preferably 6 months as determined by X-ray diffraction method.

The following non-limiting examples describe the various embodiments of the specification.

### EXAMPLE 1

| | | Quantity (mg/capsule) | |
|---|---|---|---|
| S. No | Ingredients | 1A | 1B |
| A) | Benzimidazole core | | |
| 1 | Non-pareil seeds | 90.0 | 90.0 |
| 2 | Amorphous omeprazole | 22.80 | 22.80 |
| | magnesium | | |
| 3 | Hydroxypropyl cellulose | 10.0 | 5.0 |
| 4 | Polyvinyl pyrrolidone | — | 5.0 |
| 5 | Methanol:Methylene chloride (3:7) | Qs | qs |
| B) | Separating layer | | |
| 1 | Hydroxypropyl methylcellulose | 9.46 | 9.46 |
| 2 | Polyethylene glycol 400 | 0.94 | 0.94 |
| 3 | Talc | 1.88 | 1.88 |
| 4 | Purified water | Qs | qs |
| C) | Enteric coat | | |
| 1 | Methacrylic acid copolymer Type | 33.81 | 33.81 |
| | C | | |
| 2 | Polyethylene glycol 400 | 3.38 | 3.38 |
| 3 | Talc | 12.85 | 12.85 |
| 4 | Titanium dioxide | 3.99 | 3.99 |
| 5 | Purified water | Qs | qs |
| D) | Lubrication | | |
| 1 | Talc | 0.89 | 0.89 |
| | Total | 190.0 | 190.0 |

### Process

### A. Preparation of Benzimidazole Core

1. Hydroxypropyl cellulose/Polyvinyl pyrrolidone were dissolved in a methanol: methylene chloride mixture under mechanical stirring followed by the addition of omeprazole magnesium to obtain a dispersion.
2. The dispersion of step 1 was sprayed on non-pareil seeds in a Wurster coater.

### B. Separating Layer

3. Hydroxypropyl methylcellulose was dissolved in purified water under mechanical stirring followed by the addition of polyethylene glycol and talc to obtain a coating dispersion.
4. The coating dispersion was sprayed on the benzimidazole core in a Wurster coater.

### C. Enteric Coating

5. Polyethylene glycol was dissolved in purified water under mechanical stirring followed by the addition of titanium dioxide and talc to obtain a dispersion.
6. Methacrylic acid copolymer type C was added to the dispersion of step 5 under mechanical stirring to obtain a coating dispersion.
7. The coating dispersion of step 6 was sprayed on the coated benzimidazole core in a Wurster coater.
8. The coated core of step 7 dried at 35° C.-40° C. for 15 minutes to obtain the enteric coated benzimidazole core.

### D. Lubrication

9. The enteric coated benzimidazole core was fluidized with talc in a Wurster coater for 5 minutes.
10. The core of step 1 above was dried in a vacuum tray drier at 40° C. till the loss on drying is less than 1.5% w/w.

### EXAMPLE 2

| | | Quantity | |
|---|---|---|---|
| | | mg/capsule | |
| S. No. | Ingredients | 2A | 2B |
| A | Benzimidazole Core | | |
| 1 | Non-pareil seeds (#40-60) | 30.00 | 30.00 |
| 2 | Omeprazole magnesium | 20.60 | 20.60 |
| | (Amorphous) | | |
| 3 | Hydroxypropyl cellulose | 7.20 | 7.20 |
| 4 | Polyvinyl pyrrolidone | 5.00 | 5.00 |
| 5 | Crospovidone (Kollidon CLM) | 5.00 | 3.00 |
| 6 | Magnesium oxide | — | 2.00 |
| 7 | Methanol | q.s. | q.s. |
| 8 | Methylene chloride | q.s. | q.s. |
| B | Separating layer | | |
| 1 | Hydroxypropyl methylcellulose | 6.26 | 6.26 |
| 2 | Polyethylene glycol 400 | 0.63 | 0.63 |
| 3 | Talc | 1.25 | 1.25 |
| 4 | Purified water | q.s. | q.s. |
| C | Enteric coating | | |
| 1 | Methacrylic acid copolymer | 54.90 | 54.90 |
| | Type C (Eudragit L30D 55) | | |
| | equivalent to dry polymer | | |
| 2 | Triethyl citrate | 16.50 | 16.50 |
| 3 | Glyceryl monostearate | 3.84 | 3.84 |
| 4 | Polysorbate 80 | 0.82 | 0.82 |
| 5 | Purified water | q.s. | q.s. |
| D | Lubrication | | |
| 1 | Talc | 5.00 | 5.00 |
| | Total Fill Weight | 157.00 | 157.00 |
| | Capsule Filing | | |
| | Size 2 capsule with white body and | 1 No. | 1 No. |
| | white cap | | |

### Process

### A. Preparation of Benzimidazole Core

Drug Layering
1. Non-pareil seeds were sifted through a # 40ASTM (420μ) and # 60ASTM (250μ) sieve on a mechanical vibratory sifter and the fraction retained between #40-60 was collected.
2. Hydroxypropyl cellulose-1, polyvinylpyrrolidone, crospovidone and magnesium oxide was divided into six equal parts. First part of hydroxypropyl cellulose-L, polyvinylpyrrolidone, crospovidone and magnesium oxide was sifted through a # 30 BSS (500μ) sieve on a mechanical vibratory sifter and was then dissolved in methanol and methylene chloride (1:1 ratio) under mechanical stirring.
3. Omeprazole magnesium was then divided into six equal parts. First part of Omeprazole magnesium was sifted through a # 30 BSS (500μ) sieve on a mechanical vibratory sifter. It was then dissolved in step 2 solution under mechanical stirring to obtain a dispersion.
4. The dispersion of step 3 was sprayed on non-pareil seeds in a Wurster coater.
5. The drug-loading process was continued with freshly prepared remaining five solution lots using process as given in step 2-4 to obtain the beads.
6. The beads obtained were dried at product temperature of 35° C.±5° C. for 15-30 minutes.
7. The beads were sifted using a # 25 BSS (600μ) sieve and the retention was discarded and then sifted through a # 44 BSS (355μ) sieve to remove the fines.

### B. Separating Layer

8. Hydroxypropylmethyl cellulose was sifted through a # 30 BSS (500μ) sieve on a mechanical vibratory sifter and was dissolved in purified water under mechanical stirring followed by addition of polyethylene glycol and talc to obtain a coating dispersion.
9. The drug-layered beads of step 7 were coated with the coating dispersion of step 9 to achieve weight build up of approximately 12%.
10. The coated beads were dried at product temperature of 35° C.±5° C. for 15-30 minutes.
11. The coated beads were sifted using a # 25 BSS (600μ) sieve and the retention was discarded and then sifted through s # 44 BSS (355μ) sieve to remove the fines.

### C. Enteric Coating

12. Glyceryl monostearate was dispersed in hot purified water (70° C.-80° C.) under vigorous stirring followed by the addition of Polysorbate-80.
13. Triethyl citrate was dissolved in purified water, and methacrylic acid copolymer dispersion was added to it under mechanical stirring.
14. The glycerol monostearate dispersion of step 12 was added to the dispersion of step 13 and stirred for 15-20 minutes.
15. The barrier-coated beads of step 11 were coated with dispersion of step 14.
16. The beads were dried at a product temperature of 30° C.-35° C. for 15-30 minutes.
17. Beads were sifted using a # 22 BSS (710μ) sieve and the retention was discarded and it was further sifted through a # 44 BSS (355μ) sieve to remove the fines.

### D. Lubrication

18. The beads of step 17 were dried in a vacuum tray drier at 40° C. to maintain the required LOD of beads.
19. Talc was sifted through a # 30 BSS (500μ) sieve on a mechanical vibratory sifter.
20. The delayed release coated beads of step 18 were lubricated with the material of step 20 in double cone blender for 3 to 5 minutes.

### E. Capsule Filing

21. The delayed release coated beads were filled into an approved size capsule shell.

### EXAMPLE 3

| | | Quantity (mg/tablet) | | |
|---|---|---|---|---|
| S. No | Ingredients | 3A | 3B | 3C |
| A) | Benzimidazole core | | | |
| 1 | Non-pareil seeds (#40-60) | 30.00 | 30.00 | 30.00 |
| 2 | Omeprazole magnesium | 20.60 | 20.60 | 20.60 |
| | (Amorphous) | | | |
| 3 | Hydroxypropyl cellulose | 7.20 | 7.20 | 7.20 |
| 4 | Polyvinyl pyrrolidone | 5.00 | 5.00 | 5.00 |
| 5 | Crospovidone (Kollidon CLM) | 5.00 | 5.00 | 3.00 |
| 6 | Magnesium oxide | — | — | 2.00 |
| 7 | Methanol | q.s. | q.s. | q.s. |
| 8 | Methylene chloride | q.s. | q.s. | q.s. |
| B) | Separating layer | | | |
| 1 | Hydroxypropyl methylcellulose | 6.26 | 6.26 | 6.26 |
| 2 | Polyethylene glycol 400 | 0.63 | 0.63 | 0.63 |
| 3 | Talc | 1.25 | 1.25 | 1.25 |
| 4 | Purified water | q.s. | q.s. | q.s. |
| C) | Enteric coat | | | |
| 1 | Methacrylic acid copolymer | 35.50 | 29.60 | 35.50 |
| | Type C (Eudragit L100 55) | | | |
| 2 | Hydroxypropyl methylcellulose | 23.70 | 29.60 | 23.70 |
| | Phthalate (HPMCP 55) | | | |
| 3 | Triethyl citrate | 10.70 | 10.70 | 10.70 |
| 4 | Talc | 6.16 | 6.16 | 6.16 |
| 5 | Purified water | q.s. | q.s. | q.s. |
| D) | Over coating | | | |
| 1 | Hydroxypropyl methylcellulose | 9.12 | 9.12 | 9.12 |
| 2 | Polyethylene glycol 6000 | 6.08 | 6.08 | 6.08 |
| 3 | Total Fill Weight/Tablet | 167.20 | 167.20 | 167.20 |
| E) | Compression (Tablets) | | | |
| 4 | Microcrystalline cellulose | 236.60 | 236.60 | 236.60 |
| | (Avicel 101) | | | |
| 5 | Silicified microcrystalline cellulose | 125.00 | 125.00 | 125.00 |
| | (Prosolv SMCC 90) | | | |
| 6 | Polyvinyl pyrrolidone | 45.00 | 45.00 | 45.00 |
| 7 | Crospovidone | 25.00 | 25.00 | 25.00 |
| 8 | Sodium stearyl fumarate | 1.20 | 1.20 | 1.20 |
| | Total Fill weight | 600.00 | 600.00 | 600.00 |
| F) | Coating | | | |
| 1 | Opadry AMB 80W54083 Pink | 24 mg | 24 mg | 24 mg |
| 2 | Purified water | q.s. | q.s. | q.s. |
| G) | Printing | | | |
| 1 | Opacode S-1-17734 | q.s. | q.s. | q.s. |

### Manufacturing Process

A. Benzimidazole core
1. Non-pareil seeds were sifted through a # 40ASTM (420μ) sieve and a # 60ASTM (250μ) sieve on a mechanical vibratory sifter and the fraction retained between #40-60 was collected.
2. Hydroxypropylcellulose-1, polyvinylpyrrolidone, crospovidone and magnesium oxide was divided into six equal parts. The first part of hydroxypropyl cellulose-1, polyvinylpyrrolidone, crospovidone and magnesium oxide was sifted through a # 30 BSS (500μ) sieve on a mechanical vibratory sifter and was dissolved in methanol and methylene chloride (1:1 ratio) under mechanical stirring to obtain a solution.
3. Omeprazole magnesium was also divided into six equal parts. The first part of omeprazole magnesium was sifted through a # 30 BSS (500μ) sieve on a mechanical vibratory sifter and was dissolved in the solution obtained in step 2 under mechanical stirring to form clear solution.
4. The solution of step 3 was sprayed on non-pareil seeds in a Wurster coater.
5. The drug-loading process was continued with the freshly prepared remaining five solution lots using process as given in step 2-4.
6. The beads were dried at product temperature of 35° C.±5° C. for 15-30 minutes.
7. The beads were sifted using a # 25 BSS (600μ) sieve and the retention was discarded and then sifted through a # 44 BSS (355μ) sieve to remove the fines.

### B. Separating Layer

8. Hydroxypropyl cellulose was sifted through a # 30 BSS (500μ) sieve on a mechanical vibratory sifter followed by the addition of polyethylene glycol and talc to obtain a coating dispersion.
9. The coating dispersion was sprayed on the benzimidazole core in a Wurster coater to achieve weight build up of approximately 12%.
10. The coated beads were dried at product temperature of 35° C.±5° C. for 15-30 minutes.
11. The coated beads were sifted using a # 25 BSS (600μ) sieve and the retention were discarded and then sifted through a # 44 BSS (355μ) sieve to remove the fines.

### C. Enteric Coating & D. Over Coating

12. Triethyl citrate was dissolved in purified water under mechanical stirring followed by the addition of methacrylic acid copolymer type C dispersion, hydroxypropyl methylcellulose phthalate and talc.
13. The barrier-coated beads of step 11 were coated with the dispersion of step 12 to obtain enteric coated beads.
14. The enteric coated beads were dried at a product temperature of 30° C.-35° C. for 15-30 minutes.
15. The enteric coated beads were sifted using a # 22 BSS (710μ) sieve and the retention was discarded and further sifted through a # 44 BSS (355μ) sieve to remove the fines.
16. Polyethylene glycol 6000 and hydroxypropyl methylcellulose 5 cps was dissolved in purified water under mechanical stirring.
17. The enteric-coated beads of step 15 were overcoated with coating solution of step 16 to obtain the overcoated beads
18. The overcoated beads of step 17 were dried in a vacuum tray drier at 40° C. to maintain required LOD of beads.

### E. Compression, F. Coating and G. Printing

19 Omeprazole coated beads obtained in step 18 were sifted through a mesh # 18 BSS sieve and microcrystalline cellulose, polyvinyl pyrrolidone and crospovidone were separately sifted through a # 36 BSS sieve and collected.
20. Sodium stearyl fumarate was sifted through a # 36 BSS (425μ) sieve.
21. The material of step 19 and 20 was mixed to obtain a blend in non shear blender.
22. The blend of step 21 was compressed into tablets using approved tooling.
23. Opadry Pink was dispersed in purified water under mechanical stirring to obtain a dispersion.
24. The tablets were film coated using the dispersion of Step 23 to get a target weight build up of about 4% w/w.
25. The approved inscription was printed on one side of film-coated tablets using Opacode S-1-17734 black ink.

### EXAMPLE 4

| | | Quantity (mg/ |
|---|---|---|
| S. No | Ingredients | capsule |
| A) | Benzimidazole Core | |
| 1 | Non-pareil seeds (#40/60) | 30.00 |
| 2 | Omeprazole magnesium amorphous | 20.60 |
| | (Equivalent to 20 mg of Omeprazole) | |
| 3 | Hydroxypropyl cellulose (HPC-L) | 7.20 |
| 4 | Polyvinyl pyrrolidone (PVP K30) | 5.0 |
| 5 | Methanol:Methylene chloride (1:1) | q.s |
| Total | | 62.8 mg |
| B) | Separating layer | |
| 1 | Hydroxypropyl methylcellulose (5cps) | 5.80 |
| | (Methocel E5 premium) | |
| 2 | Polyethylene glycol 400 | 0.58 |
| 3 | Talc | 1.16 |
| 4 | Purified water | q.s |
| Total | | 70.34 mg |
| C) | Enteric coat | |
| 1 | Methacrylic acid copolymer type C | 65.2 |
| | (Eudragit L30 D55) | |
| 2 | Triethyl citrate | 11.7 |
| 3 | Glyceryl monostearate | 6.5 |
| 4 | Polysorbate-80 | 1.0 |
| 5 | Purified water | q.s |
| Total | | 154.74 mg |
| D) | Over Coating | |
| 1 | Hydroxypropyl methylcellulose (5cps) | 10.8 |
| | (Methocel E5 premium) | |
| 2 | Polyethylene glycol 6000 | 4.46 |
| 3 | Purified water | q.s |
| Total | | 170.00 mg |

### Preparation of Tablets

| | | |
|---|---|---|
| 1 | Omeprazole Mg Beads | 170.0 |
| 2 | Microcrystalline cellulose (avicel PH 102) | 736.0 |
| 3 | Polyvinyl pyrrolidone USP (PVP K 30) | 60.0 |
| 4 | Crospovidone | 30.0 |
| 5 | Sodium stearyl fumarate | 2.0 |
| 6 | Colloidal silicon dioxide (Aerosil 200) | 2.0 |
| | Total | 1000.0 |
| 7 | Opadry coating | 50.0 mg |

### Process

### A. Preparation of Benzimidazole Core

1. Hydroxypropyl cellulose and polyvinyl pyrrolidone were dissolved in Methanol:Methylene chloride (1:1) under mechanical stirring followed by addition of omeprazole magnesium to obtain a clear solution.
2. The solution was sprayed on non-pareil seeds in a Wurster coater.

### B. Separating Layer

3. Hydroxypropyl methylcellulose was dissolved in purified water under mechanical stirring followed by addition of polyethylene glycol and talc to obtain a coating dispersion.
4. The coating dispersion was sprayed on the benzimidazole core in Wurster coater.

### C. Enteric Coating

5. Glyceryl monostearate was dispersed in hot purified water (70° C.) under vigorous stirring followed by Polysorbate 80.
6. Triethyl citrate was dissolved in purified water under stirring.
7. The methacrylic acid copolymer type c and glyceryl monostearate dispersion of step 5 was added to the solution of step 6 under mechanical stirring.
8. The coating dispersion of step 7 was sprayed on the barrier-coated core in a Wurster coater
9. The coated core of step 8 was dried at 35° C.-40° C. for 15 minutes to obtain the enteric-coated benzimidazole core.

### D. Over Coating

10. Hydroxypropyl methylcellulose was dissolved in purified water under mechanical stirring followed by addition of polyethylene glycol 6000.
11. The enteric coated beads were coated with solution of step 10 to achieve weight buildup of approximately 10%.

### E. Tableting

12. Microcrystalline cellulose, polyvinyl pyrrolidone, crospovidone, sodium stearyl fumarate, and colloidal silicon dioxide were blended with omeprazole beads and compressed in the form of tablets.
13. Tablets obtained were coated with opadry.

### EXAMPLE 5

| | | Quantity (mg/ |
|---|---|---|
| S. No | Ingredients | capsule |
| A) | Benzimidazole core | |
| 1 | Non-pareil seeds (#40/60) | 30.00 |
| 2 | Omeprazole magnesium amorphous | 20.60 |
| | (Equivalent to 20 mg of Omeprazole) | |
| 3 | Hydroxypropyl cellulose (HPC-L) | 7.20 |
| 4 | Polyvinyl pyrrolidone (PVP K30) | 5.0 |
| 5 | Methanol:Methylene chloride (1:1) | q.s |
| Total | | 62.8 mg |
| B) | Separating layer | |
| 1 | Hydroxypropyl methylcellulose (5cps) | 5.80 |
| | (Methocel E5 premium) | |
| 2 | Polyethylene glycol 400 | 0.58 |
| 3 | Talc | 1.16 |
| 4 | Purified water | q.s |
| Total | | 70.34 mg |
| C) | Enteric coat | |
| 1 | Methacrylic acid copolymer type C | 61.16 |
| | (Eudragit L30 D55) | |
| 2 | Triethyl citrate | 11.01 |
| 3 | Talc | 12.23 |
| 4 | Purified water | q.s |
| Total | | 154.74 mg |
| D) | Over Coating | |
| 1 | Hydroxypropyl methylcellulose USP (5cps) | 10.8 |
| | (Methocel E5 premium) | |
| 2 | Polyethylene glycol 6000 | 4.46 |
| 3 | Purified water | q.s |
| Total | | 170.00 mg |

### Preparation of Tablets

| | | |
|---|---|---|
| 1 | Omeprazole Mg Beads | 170.0 |
| 2 | Microcrystalline cellulose (avicel PH 102) | 736.0 |
| 3 | Polyvinylpyrrolidone (PVP K 30) | 60.0 |
| 4 | Crospovidone | 30.0 |
| 5 | Sodium stearyl fumarate | 2.0 |
| 6 | Colloidal silicon dioxide (Aerosil 200) | 2.0 |
| | Total | 1000.0 |
| 7 | Opadry coating | 50.0 mg |

### Process

### A. Preparation of Benzimidazole Core

1. Hydroxypropyl cellulose and polyvinyl pyrrolidone were dissolved in methanol: methylene chloride (1:1) under mechanical stirring followed by the addition of omeprazole magnesium to obtain a clear solution.
2. The solution was sprayed on non-pareil seeds in a Wurster coater.

### B. Separating Layer

3. Hydroxypropyl methylcellulose was dissolved in purified water under mechanical stirring followed by the addition of polyethylene glycol and talc to obtain a coating dispersion.
4. The coating dispersion was sprayed on the core obtained in step 2 above in Wurster coater.

### C. Enteric Coating

5. Triethyl citrate was dissolved in purified water under stirring followed by talc.
6. Methacrylic acid copolymer type C was diluted with the dispersion of step 5 under mechanical stirring.
7. The coating dispersion of step 6 was sprayed on the separating layer-coated core in Wurster coater
8. The coated core of 7 was dried at 35° C.-40° C. for 15 minutes to obtain the enteric-coated benzimidazole core.

### D. Over Coating

9. Hydroxypropyl methylcellulose was dissolved in purified water under mechanical stirring followed by the addition of polyethylene glycol 6000.
10. The enteric coated beads were coated with the solution of step 9 to achieve weight buildup of approximately 10%.

### E. Tableting

11. Microcrystalline cellulose, polyvinyl pyrrolidone, crospovidone, sodium stearyl fumarate, and colloidal silicon dioxide were blended with omeprazole beads and then compressed in the form of tablets.
12. Tablets obtained were coated with opadry.

We claim:

1. A process for the preparation of a stable oral benzimidazole composition, the process comprising the steps of:
a) preparing a benzimidazole core formed by dispersing amorphous benzimidazole compound and one or more pharmaceutically acceptable additives in one or more of non-aqueous solvents to obtain a dispersion and spraying the dispersion on a pharmaceutically acceptable inert carrier;
b) coating the core with a separating layer; and
c) coating the product of step (b) with an enteric coating,
wherein the process of preparing the benzimidazole core substantially prevents the conversion of benzimidazole compound to its crystalline form.

2. The process according to claim 1, wherein the benzimidazole compound comprises one or more of omeprazole, lansoprazole, rabeprazole, pantoprazole, leminoprazole, pariprazole, single enantiomers, pharmaceutically accepted salts, solvates or mixtures thereof.

3. The process according to claim 2, wherein the benzimidazole compound comprises omeprazole magnesium.

4. The process according to claim 1, wherein the stable oral benzimidazole composition comprises no more than about 30% by weight of the crystalline benzimidazole compound.

5. The process according to claim 1, wherein the non-aqueous solvent comprises one or more of methanol, ethanol, isopropanol, methylene chloride, acetone and mixtures thereof.

6. (canceled)

7. (canceled)

8. (canceled)

9. (canceled)

10. (canceled)

11. (canceled)

12. The process according to claim 1, wherein the separating layer comprises one or more substantially water soluble materials.

13. The process according to claim 12, wherein the substantially water-soluble material comprises one or both of a substantially water-soluble polymer and a substantially water-soluble excipient.

14. The process according to claim 13, wherein the water-soluble polymer comprises one or more of hydroxypropyl methylcellulose, hydroxypropyl cellulose, polyvinylpyrrolidone, sodium alginate, sodium carboxymethyl cellulose, and copolymer of vinylpyrrolidone and vinyl acetate.

15. The process according to claim 13, wherein the water-soluble excipient comprises one or more of lactose, mannitol, sorbitol, sucrose and glucose.
